(19) United States
(12) Patent Application Publication
(10) Pub. No.: US 2026/0294474 A1
(43) Pub. Date: Oct. 1, 2026

(54) IMPLANT DELIVERY DEVICES AND METHODS

(71) Applicants: Andrzej J. CHANDUSZKO, Franklin Lakes, NJ (US); CLEARSTREAM TECHNOLOGIES LIMITED, Franklin Lakes, NJ (US)

(72) Inventors: Andrzej J. CHANDUSZKO, Chandler, AZ (US); Hiep Q. DO, Chandler, AZ (US); Chad VAN LIERE, Phoenix, AZ (US)

(73) Assignee: CLEARSTREAM TECHNOLOGIES LIMITED, Franklin Lakes, NJ (US)

(21) Appl. No.: 19/479,156

(22) PCT Filed: Apr. 28, 2023
(86) PCT No.: PCT/US2023/020448

§ 371 Date: Oct. 27, 2025

(51) Int. Cl. A61B 17/34; A61B 17/00; A61N 5/10

(52) U.S. Cl. CPC A61B 17/3468; A61N 5/10; A61B 2017/00367; A61N 2005/1094

(57) ABSTRACT

An implant delivery device includes a shaft, a pouch, and one or more pull cords. The shaft provides delivery for implantable material and defines a delivery portion. The pouch has a proximal end and a distal end. The proximal end is positioned proximal to the delivery portion of the shaft and the distal end is removably coupled to the shaft at a position distal to the delivery portion. The one or more pull cords are pullable to release the distal end of the pouch from the shaft and open the pouch.

### TECHNICAL FIELD

The present specification generally relates to implant delivery devices and methods and, more specifically, implant delivery devices and methods for implantation of a curable material.

### BACKGROUND

During radiation treatment of cancerous tissue, radiation may at times be reflected from treated tissue or otherwise inadvertently directed to non-targeted heathy tissue. For example, in prostate cancer, radiation may be directed away from the prostate toward the rectum. To protect healthy tissue from inadvertent radiation, a layer of hydrogel may be positioned between the target tissue (e.g., the prostate) and healthy tissue (e.g., the rectum). However, during implantation where the hydrogel is initially in a liquid form, the implanted hydrogel may sometimes leak outside of a target area, which may be undesirable as such leakage to adjacent organs and anatomical structures may cause pain or discomfort, or may lead to non-uniform thickness of the spacer and potentially non-optimal radiation protection.

Accordingly, a need exists for implant delivery devices and methods for implantation of a hydrogel or other curable material, which reduces leakage.

### SUMMARY

Embodiments of the present disclosure are directed to delivery devices and methods for delivery of hydrogel or other implantable material, which results in reduced leakage, thereby reducing potential undesirable side effects and providing for improved radiation spacers.

In one embodiment, an implant delivery device includes a shaft, a pouch, and one or more pull cords. The shaft provides delivery for implantable material and defines a delivery portion. The pouch has a proximal end and a distal end. The proximal end is positioned proximal to the delivery portion of the shaft and the distal end is removably coupled to the shaft at a position distal to the delivery portion. The one or more pull cords are pullable to release the distal end of the pouch from the shaft and open the pouch.

In another embodiment, an implant deliver device includes a shaft, a pouch, and one or more pull cords. The shaft defines a plurality of delivery lumens wherein at least one delivery lumen is configured for delivery of an implantable material. The shaft defines a delivery portion. The pouch has a proximal end and a distal end, the proximal end being positioned proximal to the delivery portion of the shaft and the distal end being removably coupled to the shaft at a position distal to the delivery portion. The one or more pull cords are pullable to release the distal end of the pouch from the shaft and open the pouch.

In yet another embodiment, a method of implanting an implantable material includes advancing an implant delivery system to a target location. The implant delivery system includes a shaft, a pouch, and one or more pull cords. The shaft defines a delivery portion. The pouch has a proximal end and a distal end. The proximal end is positioned proximal to the delivery portion of the shaft and the distal end is removably coupled to the shaft at a position distal to the delivery portion. The method further includes delivering the implantable material to the target location via the delivery portion of the shaft, wherein delivering the implantable material fills at least a portion of the pouch surrounding the delivery portion of the shaft, curing the implantable material thereby providing cured implantable material, pulling the one or more pull cords to release the distal end of the pouch from the shaft and open the pouch, and retracting the pouch in a proximal direction from the cured implantable material.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a delivery device for delivering an implantable material including a shaft, a pouch, and one or more pull cords, according to one or more embodiments shown and described herein;

FIG. 1B schematically illustrates a longitudinal cross-section of the delivery device of FIG. 1A, accordingly to one or more embodiments shown and described herein;

FIG. 1C schematically illustrates removal of the one or more pull cords of FIG. 1A thereby releasing the pouch from an implantable material, according to one or more embodiments shown and described herein;

FIG. 2A schematically illustrates an incision formed at a target site, according to one or more embodiments shown and described herein;

FIG. 2B schematically illustrates insertion and expansion of the delivery device of FIG. 1A into the incision of FIG. 2A, according to one or more embodiments shown and described herein;

FIG. 2C schematically depicts a cross-section of the pouch of the delivery device of FIG. 2B filled with implantable material, according to one or more embodiments shown and described herein;

FIG. 2D schematically depicts removal of the one or more pullcords from the delivery device of FIG. 2C, according to one or more embodiments shown and described herein;

FIG. 2E schematically depicts removal of the delivery device of FIG. 2D from the incision while leaving cured implantable material, according to one or more embodiments shown and described herein;

FIG. 3A schematically depicts an embodiment of a delivery device prior to insertion into an incision, according to one or more embodiments shown and described herein;

FIG. 3B schematically depicts a cross section of the delivery device of FIG. 3A including a shaft, a pouch, and one or more pull cords delivered within an incision and filled with implantable material, according to one or more embodiments shown and described herein;

FIG. 3C schematically depicts removal of the one or more pull cords through the shaft of FIG. 3B, according to one or more embodiments shown and described herein;

FIG. 3D schematically depicts withdrawal of the delivery device of FIG. 3C while leaving cured implantable material within the incision, according to one or more embodiments shown and described herein; and

FIG. 4 schematically illustrates a longitudinal cross-section of yet another embodiments of a delivery device, according to one or more embodiments shown and described herein.

### DETAILED DESCRIPTION

The present disclosure is directed to implant delivery devices and methods for implanting an implantable material. For example, embodiments as provided herein may be useful for implantation of hydrogel materials for use as, for example, radiation spacers. Radiation spacers may be used to shield healthy tissue from radiation directed toward diseased or cancerous tissue. For example, radiation therapy for prostate cancer can take the form of placement of brachytherapy seeds into the prostate or external beam radiation therapy. Scatter radiation from these techniques, however, provides a significant risk to adjacent organs. As radiation therapy technology and administration techniques have improved, a significant limiting factor to dose administration is the risk of rectal toxicity from scatter radiation. Radiation spacers such as provided herein provide shielding to reduce the risk of radiation impacting non-targeted tissue.

In one embodiment, radiation spacers may be useful in the separation of healthy rectal tissue from treated prostate tissue, for example, during radiation treatment. Because radiation dose is inversely proportional to the distance squared, separating the prostate from the rectum can significantly lower the non-targeted tissue's exposure to radiation. The radiation spacer can also act as a radiation shield. In one embodiment, an implant delivery device includes a shaft for delivery of implantable material, a pouch that receives the implantable material, and one or more pull cords. The implantable material may be allowed to cure within the pouch and then after sufficient curing, the one or more pull cords are pullable to release the pouch, such that the pouch may be withdrawn from the cured implantable material. Accordingly, implantable material may be maintained in position during curing, thereby leading to reduced leakage of implantable material from the target delivery space and leading to reduced side effects, as described above, and, in embodiments, improved radiation spacers.

As used herein, the term “proximal” means closer to or in the direction of an origin of an element, such as a handle or other user manipulated portion of a device. The term “distal” means further from the origin, such as toward a tip of the element.

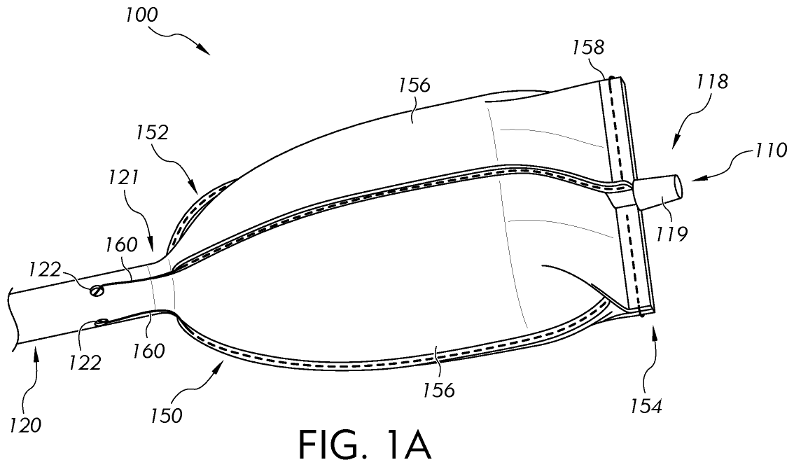

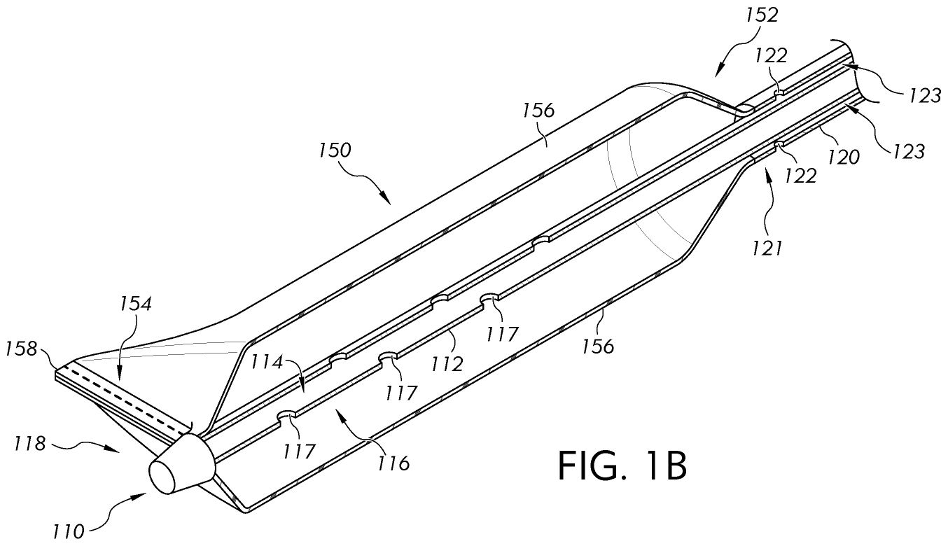

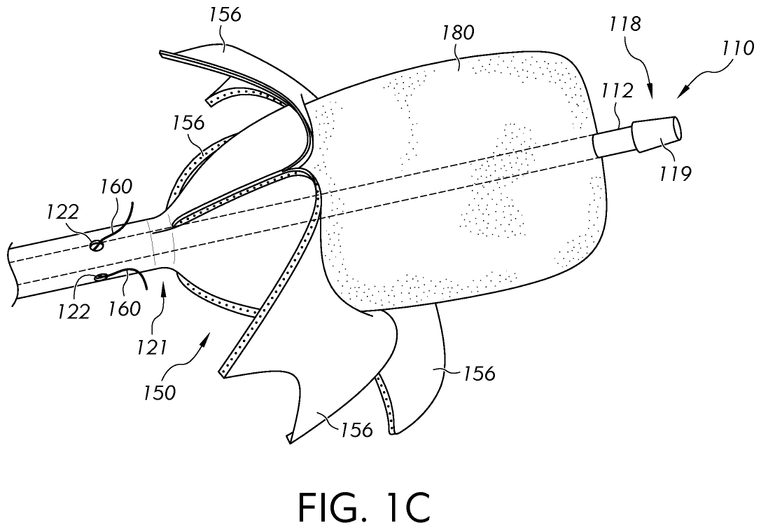

Referring to FIGS. 1A-1C, an implant delivery device 100 is generally depicted. The implant delivery device 100 generally includes a shaft 110, a pouch 150, and one or more pull cords 160. In embodiments, the implant delivery device 100 may further include a sheath 120. In some embodiments, the implant delivery device 100 may further include a pump, syringe, or similar device for delivery of implantable material 180 into the pouch 150 via the shaft 110, for example. The implant delivery device 100 may include a greater or fewer number of components without departing from the scope of the present disclosure.

Referring specifically to FIG. 1B, a longitudinal cross-section (e.g., along a centerline of the shaft 100) of the delivery device 100 is depicted. As illustrated, the shaft 110 may have an elongate body 112 that defines one or more lumens 114 extending therethrough to allow for transport of implantable material 180 (illustrated in FIG. 1C for example) within the elongate body 112, from a proximal end (not depicted) of the elongate body 112 toward a distal end 118 of the elongate body 112. The shaft 110 may be formed of any flexible or inflexible material, such as pebax, nylon, polyurethane, polyethylene terephthalate (PET), thermoplastic elastomers, PEEK, etc.

The shaft 110, such as the elongate body 112, defines a delivery portion 116, such as at or adjacent to the distal end 118 of the shaft 110. The delivery portion 116 may define an array of implantable material inlets 117. The array of implantable material inlets 117 may include any number of inlets such as two or more, three or more, five or more, etc. The inlets may have any shape such as round, oval, rectangular, etc. In embodiments, the array of implantable material inlets 117 may be arranged radially around the delivery portion 116 so as to dispense the implantable material 180 at various radial positions about the delivery portion 116. Accordingly, material may flow substantially evenly through one or more lumens 114 and out various inlets, or portions thereof, to fill the pouch 150. In some embodiments, the size of each of the inlets of the array of implantable material inlets 117 may vary from one another. For example, the size of the inlets may be arranged along a gradient such as smaller to larger in a proximal to distal direction. Such arrangement may reduce resistance within the shaft 110 during material delivery and ensure more even diffusion of material from the shaft 110.

At the distal end 118 of the shaft 110 may be a tip 119. The tip 119 may be rounded, such as to be atraumatic. In embodiments, the tip 119 may be tapered to aid in insertion of the tip 119 into a target location. In some embodiments, the tip 119 may be a cap affixed over the distal end 118 of the shaft 110.

Referring collectively to FIGS. 1A-1C, the pouch 150 may have a proximal end 152 and a distal end 154. The proximal end 152 may be positioned proximal to the delivery portion 116 of the shaft 110. The distal end 154 may be removably coupled to the shaft 110 at a position distal to the delivery portion 116. That is, the distal end 154 may be removably coupled to the shaft 110 around the tip 119, as depicted. Accordingly, during implantable material 180 delivery, pouch 150 may span the delivery portion 116, such that implantable material 180 may be delivered into the pouch 150, as opposed to outside of the pouch 150. That is, during delivery of the implantable material 180, the implantable material 180 may fill the pouch 150, instead of leaking from the target area.

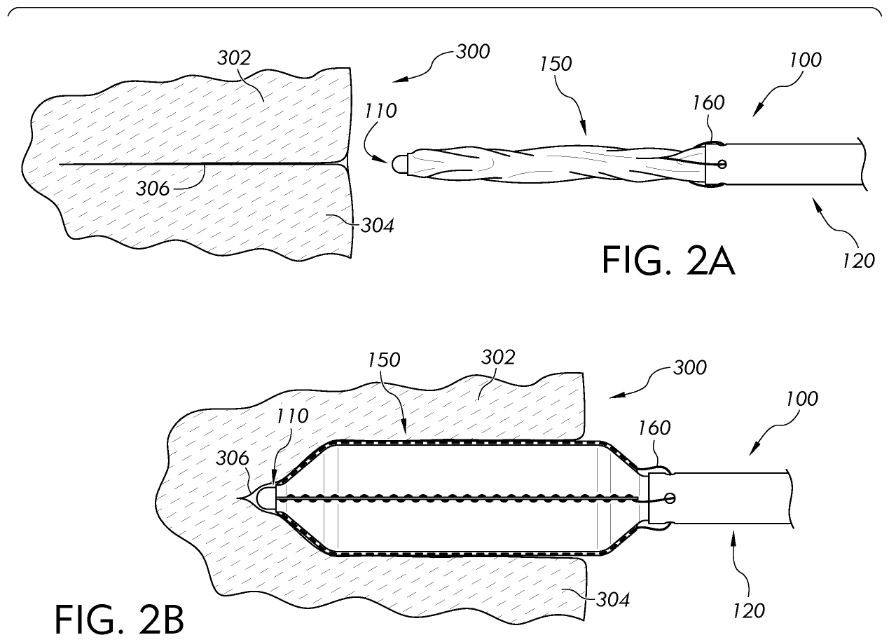

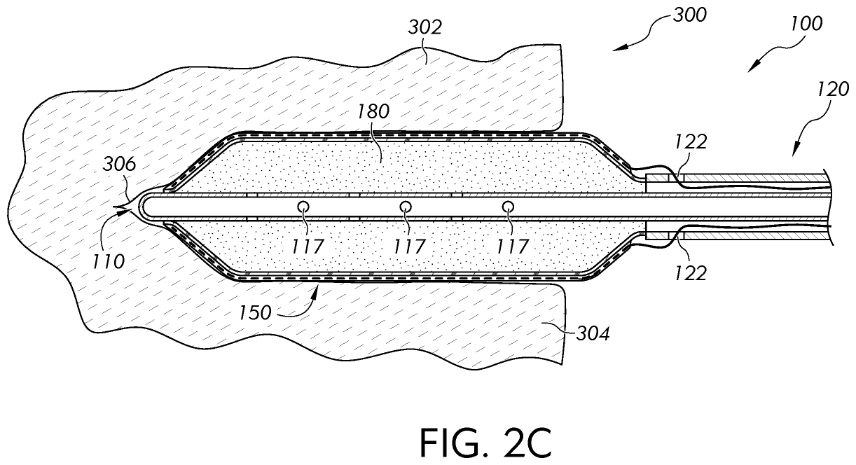

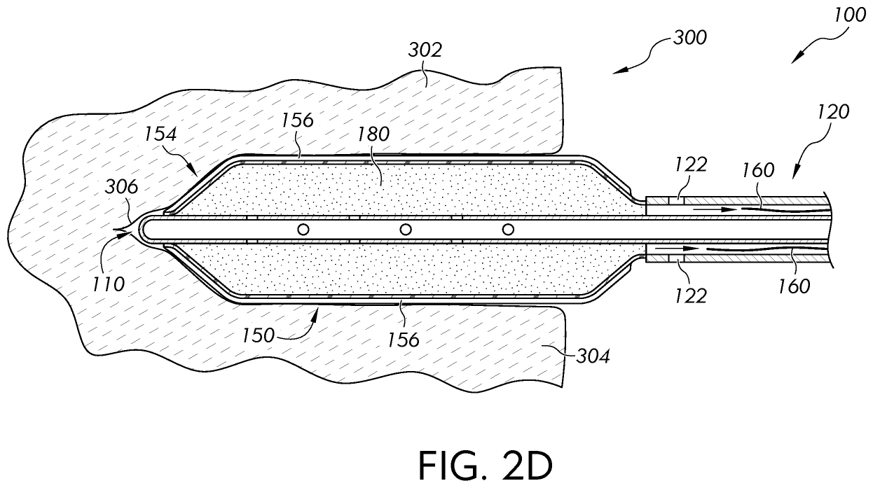

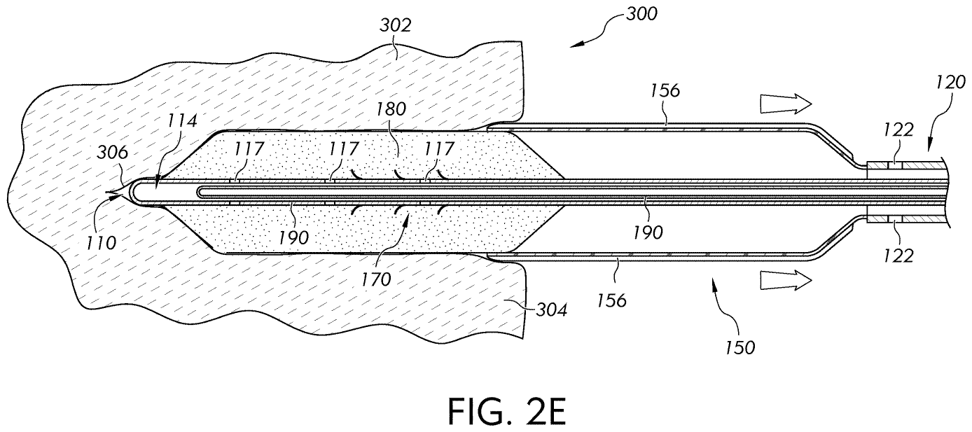

During delivery of the implantable material 180 to a target site, the pouch 150 may be wrapped or folded around the shaft 110 to have an initial reduced profile (such as depicted in FIG. 2A). As the implantable material 180 is delivered into the pouch 150, the pouch 150 may expand (e.g., unfold) to accommodate the implantable material 180 (such as depicted in FIG. 2C).

The pouch 150 may be formed of a number of materials, such as, but not limited to ePTFE, urethane, silicone, polyoxymethylene (POM), or the like. The pouch 150 may be formed of a plurality of sections 156 (such as two or more sections, three or more sections, four or more sections, etc.), and may also be referred to as radial sections. For example, each section 156 may extend circumferentially about the shaft such as to collectively form a volumetric space between the shaft 110 and the pouch 150. The sections 156 may extend longitudinally along the shaft 110 and may be at least partially coupled to one another via, for example the one or more pull cords 160. For example, the sections 156 may be sewn together, such as with a chain stitch, zip stitch, or the like (for example a removable stitch having a pullable thread that upon pulling the thread pulls out the stitch similar to that used in animal feed bags). Accordingly, as the one or more pull cords 160 are pulled the plurality of sections 156 may at least partially come apart from one another. By allowing the plurality of sections 156 to separate via pulling of the one or more pull cords 160, it may be easier to release the pouch 150 from the cured implantable material 180.

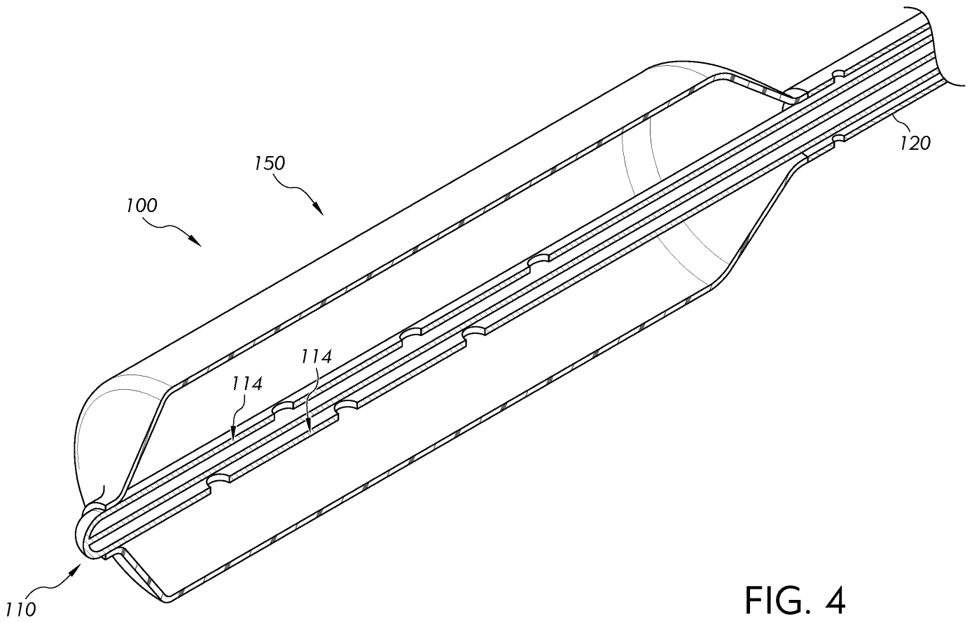

As illustrated in FIGS. 1A and 1B, the distal end 118 of the pouch 150 is removably coupled to the shaft 110 at a position distal to the delivery portion 116. For example, the distal end 118 of the pouch 150 may be coupled to distal end 118 of the shaft 110 via the one or more pull cords 160. For example, the one or more pull cords 160 may sew the end of the pouch 150, such as the ends of the sections 156, shut and hold the pouch 150 in place relative to the distal end 118 of the shaft 110. Accordingly, when the one or more pull cords 160 are pulled, the distal end 118 of the pouch 150 may open and be released from the shaft 110. In the depicted embodiment, a seam 158 at the distal end 154 of the pouch 150 is depicted as being transverse to a longitudinal axis of the shaft 110. However, in other embodiments, the seam 158 may not be oriented as depicted. Instead, the pouch 150 may taper toward the distal end 118 of the shaft 110 (such as depicted in FIG. 4).

FIG. 1C illustrates the one or more pull cords 160 having been removed. With the one or more pull cords 160 removed, the sections 156 of the pouch 150 may be separated from the distal end 154 of the shaft 110 and may also at least partially separate from one another longitudinally as depicted. Such unfastening of the sections 156, may allow for the pouch 150 to be more easily pulled from the cured implantable material 180.

In embodiments, and to aid in removal of the pouch 150, an inner surface and/or an outer surface) of the pouch 150 may be coated with one or more coatings to aid in removal of the pouch 150 from the cured implantable material 180. For example, the inside of the pouch 150 may be coated with hydrophobic materials (e.g., PVDF-HFP), self-lubricated silicone, Acetal, or the like.

In embodiments, a proximal end 152 of the pouch 150 may be coupled to the sheath 120. For example, the sheath 120 includes a sheath distal end 121 and the proximal end 152 of the pouch 150 is coupled to the sheath 120 distal end 118 such as via adhesive, welding, or any conventional means. The sheath 120 may define a sheath lumen 123 and the shaft 110 may be positioned within the sheath 120 lumen. The sheath 120 may be formed of any suitable material such as, but not limited to pebax, nylon, polyurethane, polyethylene terephthalate (PET), and thermoplastic elastomers, PEEK, etc.

Formed in the sheath 120 distal end 118, may be one or more pull cord holes 122 which are sized to receive the one or more pull cords 160. For example, each pull cord may extend through a separate pull cord hole, so as to be positioned with the sheath 120 lumen in a proximal direction. The one or more pull cord holes 122 may include a plurality of pull cord holes (e.g., two or more, three or more, etc.) for receiving a pull cord therethrough.

As noted above, the one or more pull cords 160 may be sewn into the pouch 150 to couple the sections 156 of the pouch 150 to one another and/or to seal the distal end 118 of the pouch 150. The one or more pull cords 160 may be routed from the pouch 150 through the one or more pull cord holes 122 so as to be positioned within the sheath lumen 123. The one or more pulls cords 160 may be manipulable (e.g., pullable) by a user at a proximal end of the device or sheath 120 to open the pouch 150. The one or more pull cords 160 may be formed of any suitable material such as, but not limited to, silk, polypropylene, etc.

Referring now to FIGS. 2A-2E a method of implanting an implantable material 180 at a target site 300 with the implant delivery device 100 is generally depicted. A target site 300 for implant delivery may include any site where it may be desirable to implant an implantable material 180. For example, the implantable material 180 may include a curable hydrogel such as, but not limited to, Chitosan, alginate, gelatin, polyethylene glycol hydrogels, PLGA-PEG hydrogel, PLA-PEG hydrogel, PCL-PEG hydrogel, Poly(N-isopropylacrylacrylamide) (PolyNIPAm), etc. For example, in embodiments it may be desirable to implant a spacer or protective layer between cancerous and otherwise healthy tissue, such as between a cancerous prostate and a healthy rectum. During such procedures, an incision 306 may be made at a target site 300 between a first tissue 302 (e.g., the rectum) and a second tissue 304 (e.g., the prostate). The incision 306 may be formed via any means such as via hydrodissection, laser cutting, a blade, or the like. A dilator (not depicted) may be positioned within the dilated to enlarge the incision 306 if needed and prior to placement of the implant delivery device 100.

Referring now to FIG. 2A, the implant delivery device 100 is illustrated as being in a reduced profile. That is the pouch 150 is uninflated with implantable material 180. While uninflated, the pouch 150 may be wrapped around the shaft 110 for ease of insertion into the incision 306. Once positioned within the incision, as illustrated in FIG. 2B, the pouch 150 may be inflated or filled with the implantable material 180 (shown in FIG. 2C), which may expand the incision 306. FIG. 2C illustrates a cross-section of the implant delivery device 100 with the implantable material 180 within the pouch 150. For example, the implantable material 180 may be delivered through the shaft 110 (e.g., via a pump, syringe, etc.) and directed through the one or more implantable material inlets 117 into the pouch 150. It is noted that the pouch 150 may have any cross-sectional shape upon expansion. For example, the pouch 150 may be round, oblong, square, rectangular, or the like. Once the pouch 150 is sufficiently filled, such as may be determined by no further implantable material 180 being able to be advance into the pouch 150 or using a premeasured amount of implantable material, the implantable material 180 may be allowed to cure within the pouch 150 (such as for thirty seconds or more, one or more minutes, five or more minutes, ten or more minutes, or any suitable amount of time depending on the implantable material 180).

Referring now to FIG. 2D, after curing, the one or more pull cords 160 may be pulled thereby opening the distal end 154 of the pouch 150. As noted above, pulling the one or more pull cords 160 may further release the sections 156 of the pouch 150 from one another along the longitudinal direction. The sheath 120 and pouch 150 may then be retracted. For example, an operator may pull the sheath 120 and due to the coupling between the sheath 120 and the pouch 150, may similarly pull the pouch 150. In the present embodiment, the shaft 110 may remain in place as the sheath 120 and pouch 150 are withdrawn. In some embodiments, the shaft 110 may have one or more anchoring features 170 (e.g., injection holes, barbs, surface texture, shaft geometry (e.g., corkscrew shape), or the like), which may aid in holding the implanted material in place during withdrawal of the pouch 150 and sheath 120. In some embodiments, during removal of the shaft 110, a release member 190 may be advanced through the shaft lumen 114 or over the shaft 110 to sever any cured implantable material 180 which may otherwise be holding the shaft 110 in place. In some embodiments, saline or other releasing agent may be delivered via the shaft lumen 114 and through the one or more implantable material inlets 117 to aid in release of the shaft 110. Accordingly, after curing and/or withdrawal of the pouch 150, the shaft 110 may then be removed. In some embodiments, twisting the shaft 110 with removal may aid in removing the shaft 110 from the implantable material 180. For example, twisting of the shaft 110 may also assist is dislodging the one or more anchoring features 170 from the implantable material 180.

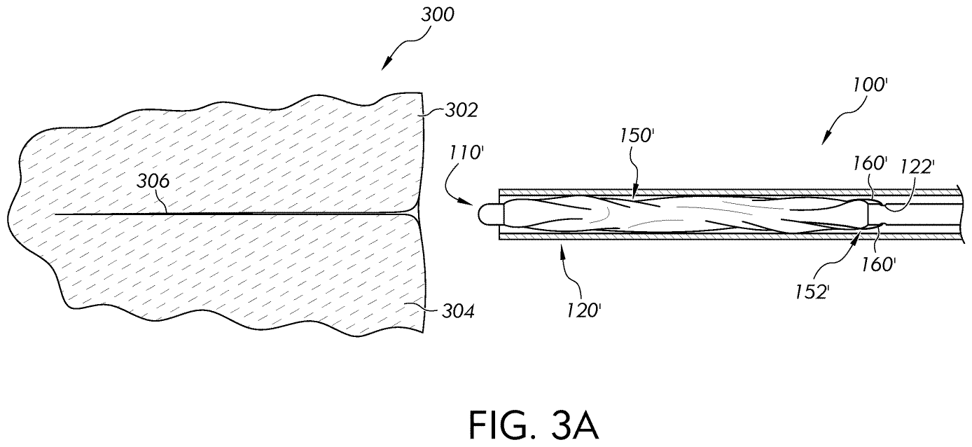

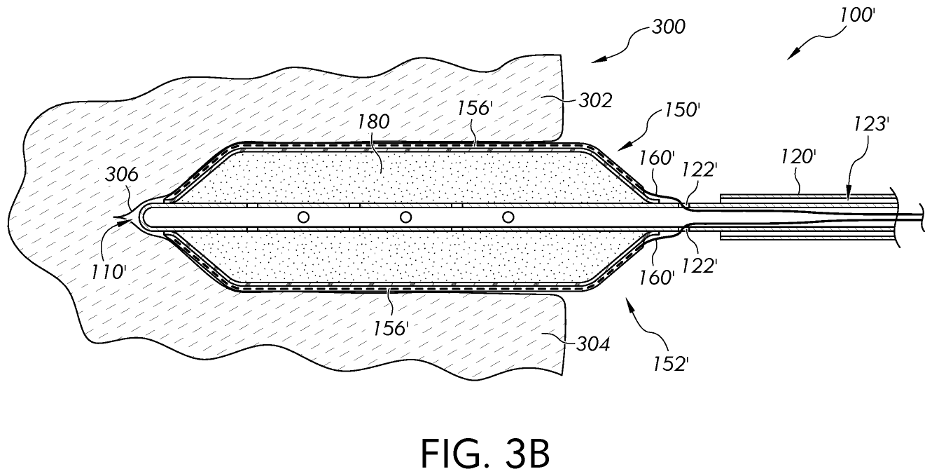

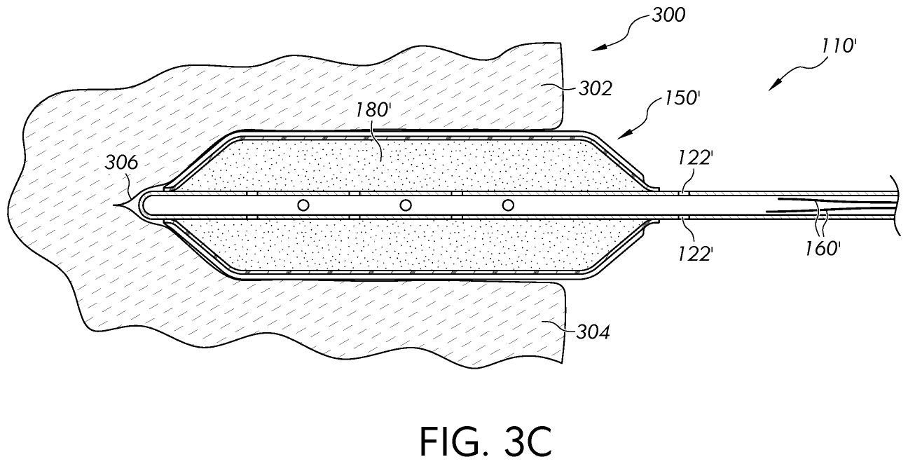

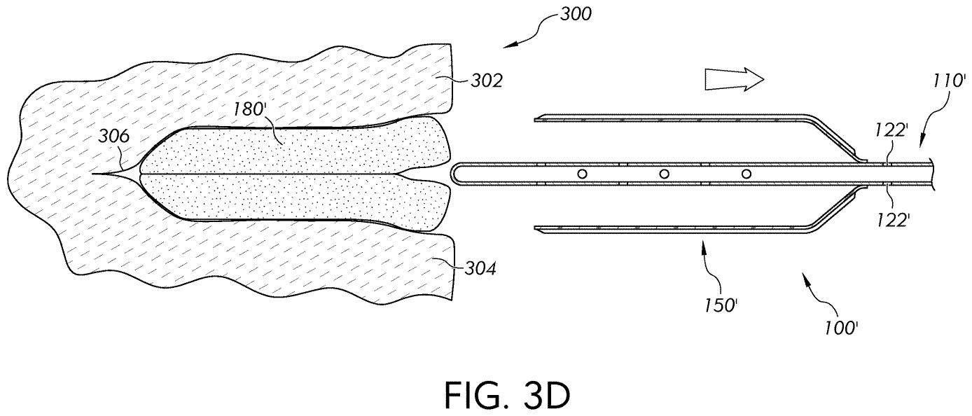

FIGS. 3A-3D illustrate an alternative embodiment of an implant delivery device 100′. The implant delivery device 100′ is substantially similar to the implant delivery device 100 described above. Accordingly, the above description applies equally to the embodiment depicted in FIG. 3A-3D unless otherwise noted or apparent. In particular, in the present embodiment, the implant delivery device 100′ similarly includes a shaft 110′, a pouch 150′, one or more pull cords 160′, and a sheath 120′, though a greater or fewer number of components of contemplated and possible. However, in the present embodiment, the pouch 150′ is not coupled to the sheath 120′ at the proximal end 152′ of the pouch 150′, but is instead coupled to the shaft 110′ at the proximal end 152′ of the pouch 150′. In the present embodiment, the shaft 110′ may include one or more pull cord openings 122′ such that the one or more pull cords 160′ are routed through the lumen 114 of the shaft 110′. It is however, contemplated that there might not be any pull cord openings and, instead, the one or more pull cords 160 could simply be routed through the lumen 123′ of the sheath 120′ and run outside of and adjacent to the shaft 110′.

Similar to the description above, FIGS. 3A-3D illustrate a method of delivering an implantable material 180 into an incision 306, such as described above. In the present embodiment, sheath 120′ may be initially positioned over the pouch 150′, which may assist in maintaining the pouch 150′ in a low profile orientation. In embodiments, the sheath 120 may be positioned along within the pouch 150′ in the incision 306 and then the sheath 120′ withdrawn relative to the pouch 150′, such as illustrated in FIG. 3B. Once positioned within the incision 306′, as in the embodiment above, the implantable material 180 may be delivered via the shaft 110′ and into the pouch 150′ and then allowed to cure. Once cured, the one or more pull cords 160′ may be manipulated by a user to release the pouch 150′ and/or the plurality of sections 156′ from one another. In the present embodiment, because the shaft 110′ is attached to the pouch 150′, the shaft 110′ and pouch 150′ may be withdrawn simultaneously. As noted above, in various embodiments, lubricants, removal devices or the like, may be used to aid in removal of the shaft 110′ from the cured implantable material 180. For example, a removal device, such as illustrated in FIG. 2D may be advance within the lumen of the shaft 110 to cut a cured material in the lumen, which may resist removal of the shaft 110.

Referring now to FIG. 4, in any of the embodiments described herein, the shaft 110 may define multiple lumens 114. For example, the shaft 110 may define a plurality of lumens 114. A portion of the plurality of lumens 114 may be used for delivering implantable material 180. A separate portion of the plurality of lumens 114 may be used to deliver a release fluid such a saline, water or the like, to aid in lubricating and releasing the shaft 110 from the implanted material. However and as noted above, in some embodiments, the release fluid may be pushed through the same lumen of the implantable material 180 after the implantable material 180 cures. In some embodiments, a small volume of release fluid might be used to flush hydrogel out of the sheath before it cures. In some embodiments, the multiple lumens may have different sizes. For example, the shaft 110 may define two lumens, one larger lumen for delivering of implantable material and a smaller lumen for lubricating fluid. Separate lumens may also be used for maintaining separation of components of the hydrogel prior to delivery of the hydrogel to the target site. Such may prevent gelling or curing of the hydrogel within the lumen.

Embodiments of the present disclosure may be further described with respect to the following numbered clauses:
1. An implant delivery device comprising: a shaft for delivery of an implantable material, the shaft defining a delivery portion; a pouch having a proximal end and a distal end, the proximal end being positioned proximal to the delivery portion of the shaft and the distal end being removably coupled to the shaft at a position distal to the delivery portion; and one or more pull cords pullable to release the distal end of the pouch from the shaft and open the pouch.
2. The implant delivery device of clause 1, wherein the delivery portion of the shaft defines an array of implantable material inlets.
3. The implant delivery device of any preceding clause, further comprising a sheath defining a lumen and surrounding the shaft.
4. The implant delivery device of clause 3, wherein the sheath comprises a sheath distal end and the proximal end of the pouch is coupled to the sheath distal end; or the sheath proximal end is coupled to the shaft.
5. The implant delivery device of any preceding clause, wherein the pouch comprises a plurality of sections coupled to one another and positioned around the shaft.
6. The implant delivery device of clause 5, wherein the one or more pull cords are configured to release the plurality of sections at least partially from one another when pulled.
7. The implant delivery device of any preceding clause, wherein the delivery portion of the shaft comprises one or more anchoring features configured to anchor the implantable material in place during withdrawal of the pouch.
8. An implant delivery device comprising: a shaft defining a plurality of delivery lumens wherein at least one delivery lumen is configured for delivery of an implantable material, the shaft defining a delivery portion; a pouch having a proximal end and a distal end, the proximal end being positioned proximal to the delivery portion of the shaft and the distal end being removably coupled to the shaft at a position distal to the delivery portion; and one or more pull cords pullable to release the distal end of the pouch from the shaft and open the pouch.
9. The implant delivery device of clause 8, wherein the delivery portion of the shaft defines an array of inlets fluidically coupled to at least one of delivery lumen of the plurality of delivery lumens.
10. The implant delivery device of clause 8 or 9, further comprising a sheath defining a lumen and surrounding the shaft.
11. The implant delivery device of clause 10, wherein the sheath comprises a sheath distal end and the proximal end of the pouch is coupled to the sheath distal end, wherein the sheath and the pouch are retractable relative to the shaft upon release of the distal end of the pouch from the shaft.
12. The implant delivery device of any of clauses 8 to 11, wherein the pouch comprises a plurality of sections coupled to one another and positioned around the shaft.
13. The implant delivery device of clause 12, wherein the plurality of sections include at least four sections at least partially releasably coupled to one another via the one or more pull cords.
14. The implant delivery device of clause 12 or 13, wherein the one or more pull cords are configured to release the plurality of sections at least partially from one another when pulled.
15. The implant delivery device of any of clauses 8 to 14, wherein the delivery portion of the shaft comprises one or more anchoring features configured to anchor the implantable material as the pouch is withdrawn.
16. A method of implanting an implantable material, the method comprising: advancing an implant delivery system to a target location, the implant delivery system comprising: a shaft defining a delivery portion; a pouch having a proximal end and a distal end, the proximal end being positioned proximal to the delivery portion of the shaft and the distal end being removably coupled to the shaft at a position distal to the delivery portion; and one or more pull cords; delivering the implantable material to the target location via the delivery portion of the shaft, wherein delivering the implantable material fills at least a portion of the pouch surrounding the delivery portion of the shaft; curing the implantable material thereby providing cured implantable material; pulling the one or more pull cords to release the distal end of the pouch from the shaft and open the pouch; and retracting the pouch in a proximal direction from the cured implantable material.
17. The method of clause 16, further comprising removing the shaft from the cured implantable material by withdrawing the shaft proximally from the cured implantable material.
18. The method of any preceding clause, wherein removing the shaft from the cured implantable material further comprises delivering a release agent from the delivery portion to release the cured implantable material from the shaft.
19. The method of any preceding clause, wherein removing the shaft from the cured implantable material further comprises advancing a release member through a lumen of the shaft to release the cured implantable material from the shaft.
20. The method of any preceding clause, wherein removing the shaft from the cured implantable material comprises rotating the shaft relative to the cured implantable material.
21. The implant delivery device of any of clauses 1-15, wherein the shaft defines a plurality of lumens.

It should now be understood that embodiments of the present disclosure and directed to a delivery device for the delivery of implantable material. For example, embodiments as provided herein may be useful for implantation of hydrogel materials for use as, for example, radiation spacers. In embodiments, implantable material may be allowed to cure within a pouch and then after sufficient curing, the one or more pull cords are pullable to release the pouch, such that the pouch may be withdrawn from the cured implantable material. Accordingly, implantable material may be maintained in position during curing, thereby leading to reduced leakage of implantable material from the target delivery space. Leading to reduced waste and mess.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. An implant delivery device comprising:
a shaft for delivery of an implantable material, the shaft defining a delivery portion;
a pouch having a proximal end and a distal end, the proximal end being positioned proximal to the delivery portion of the shaft and the distal end being removably coupled to the shaft at a position distal to the delivery portion; and
one or more pull cords pullable to release the distal end of the pouch from the shaft and open the pouch.

2. The implant delivery device of claim 1, wherein the delivery portion of the shaft defines an array of implantable material inlets.

3. The implant delivery device of claim 1, further comprising a sheath defining a lumen and surrounding the shaft.

4. The implant delivery device of claim 3, wherein the sheath comprises a sheath distal end and the proximal end of the pouch is coupled to the sheath distal end.

5. The implant delivery device of claim 1, wherein the pouch comprises a plurality of sections coupled to one another and positioned around the shaft.

6. The implant delivery device of claim 5, wherein the one or more pull cords are configured to release the plurality of sections at least partially from one another when pulled.

7. The implant delivery device of claim 1, wherein the delivery portion of the shaft comprises one or more anchoring features configured to anchor the implantable material in place during withdrawal of the pouch.

8. An implant delivery device comprising:
a shaft defining a plurality of delivery lumens wherein at least one delivery lumen is configured for delivery of an implantable material, the shaft defining a delivery portion;
a pouch having a proximal end and a distal end, the proximal end being positioned proximal to the delivery portion of the shaft and the distal end being removably coupled to the shaft at a position distal to the delivery portion; and
one or more pull cords pullable to release the distal end of the pouch from the shaft and open the pouch.

9. The implant delivery device of claim 8, wherein the delivery portion of the shaft defines an array of inlets fluidically coupled to at least one of delivery lumen of the plurality of delivery lumens.

10. The implant delivery device of claim 8, further comprising a sheath defining a lumen and surrounding the shaft.

11. The implant delivery device of claim 10, wherein the sheath comprises a sheath distal end and the proximal end of the pouch is coupled to the sheath distal end, wherein the sheath and the pouch are retractable relative to the shaft upon release of the distal end of the pouch from the shaft.

12. The implant delivery device of claim 8, wherein the pouch comprises a plurality of sections coupled to one another and positioned around the shaft.

13. The implant delivery device of claim 12 wherein the plurality of sections include at least four sections at least partially releasably coupled to one another via the one or more pull cords.

14. The implant delivery device of claim 12, wherein the one or more pull cords are configured to release the plurality of sections at least partially from one another when pulled.

15. The implant delivery device of claim 8, wherein the delivery portion of the shaft comprises one or more anchoring features configured to anchor the implantable material in place as the pouch is withdrawn.

16. A method of implanting an implantable material, the method comprising:
advancing an implant delivery system to a target location, the implant delivery system comprising:
a shaft defining a delivery portion;
a pouch having a proximal end and a distal end, the proximal end being positioned proximal to the delivery portion of the shaft and the distal end being removably coupled to the shaft at a position distal to the delivery portion; and
one or more pull cords;
delivering the implantable material to the target location via the delivery portion of the shaft, wherein delivering the implantable material fills at least a portion of the pouch surrounding the delivery portion of the shaft;
curing the implantable material thereby providing cured implantable material;
pulling the one or more pull cords to release the distal end of the pouch from the shaft and open the pouch; and
retracting the pouch in a proximal direction from the cured implantable material.

17. The method of claim 16, further comprising removing the shaft from the cured implantable material by withdrawing the shaft proximally from the cured implantable material.

18. The method of claim 16, wherein removing the shaft from the cured implantable material further comprises delivering a release agent from the delivery portion to release the cured implantable material from the shaft.

19. The method of claim 16, wherein removing the shaft from the cured implantable material further comprises advancing a release member through a lumen of the shaft to release the cured implantable material from the shaft.

20. The method of claim 16, wherein removing the shaft from the cured implantable material comprises rotating the shaft relative to the cured implantable material.
